(19) United States
(12) Patent Application Publication
(10) Pub. No.: US 2023/0132326 A1
(43) Pub. Date: Apr. 27, 2023

(54) DATA PROCESSING METHOD, DATA PROCESSING SYSTEM, AND COMPUTER PROGRAM

(71) Applicant: SHIMADZU CORPORATION, Kyoto (JP)

(72) Inventors: Yuichiro FUJITA, Kyoto (JP); Akira NISHIO, Kyoto (JP); Davide Giovanni VECCHIETTI, Kyoto (JP)

(21) Appl. No.: 17/951,553

(22) Filed: Sep. 23, 2022

(30) Foreign Application Priority Data
Oct. 25, 2021 (JP) 2021-173643

(51) Int. Cl. G01N 33/68; G01N 30/72

(52) U.S. Cl. CPC G01N 33/6848; G01N 30/72

(57) ABSTRACT

A data processing method for creating simulation data of a three-dimensional chromatogram for a sample containing a plurality of components. The data processing method includes a data preparing step of preparing chromatogram data and spectrum data of each of a plurality of the components, a parameter determining step of determining a parameter including a ratio of concentration of a plurality of the components in the sample to each other and separation degree of peaks of a plurality of the components on a chromatogram from each other, a data adjusting step of adjusting a peak area and a peak position of chromatogram data of each of a plurality of the components based on the parameter determined in the parameter determining step, and a matrix product step of calculating matrix product of a spectrum data group including spectrum data of each of a plurality of the components and a chromatogram data group including chromatogram data of each of a plurality of the components adjusted in the data adjusting step, and creating simulation data of a three-dimensional chromatogram for the sample based on a result of the calculation.

### BACKGROUND OF THE INVENTION

### 1. Field of the Invention

The present invention relates to a data processing method, a data processing system, and a computer program.

### 2. Description of the Related Art

In a liquid chromatograph (hereinafter referred to as LC-PDA) using a multichannel detector such as a photodiode array (PDA) detector, three-dimensional chromatogram data having three dimensions of time, wavelength, and signal intensity (absorbance) can be obtained by continuously acquiring an absorption spectrum of a sample eluted from an analysis column.

In a case where a target component in a sample is quantified using a liquid chromatograph, in general, a chromatogram is created using a wavelength at which the absorbance of the target component is the largest, and an area value of a peak of the target component is obtained on the chromatogram to perform quantification. However, a sample may contain an impurity other than the target component, and a peak of the impurity may overlap a peak of the target component on the chromatogram. In such a case, it is not possible to obtain a peak area value of the target component or the impurity if a plurality of peaks overlap each other, and a quantification result cannot be obtained. For this reason, it is necessary to separate a plurality of components whose peaks overlap each other on the chromatogram from each other.

As a method for separating peaks of a plurality of components overlapping each other, there is a method of estimating a chromatogram of each component by applying a model function (peak model) such as an exponential modified gaussian (EMG) function to a waveform of an actual chromatogram (see WO 2016/035167 A).

### SUMMARY OF THE INVENTION

When impurities having concentration of several 1/100 to several 1/1000 with respect to concentration of a main component are mixed in a sample and the peaks of these components overlap each other, in the peak separation method using the model function as described above, a peak area value of a low-concentration component (impurity) is buried in an error between an actual component peak of the low-concentration component and the model function simulating the actual component peak, and a correct shape and size of a peak of the low-concentration component cannot be accurately determined.

There is a demand for analyzing a sample containing a plurality of components under such an analysis condition that each component peak is separated from other components at desired separation degree. However, as described above, in a case where peaks of a plurality of components having a very large concentration difference overlap each other, a shape and size of a peak of low-concentration component cannot be accurately estimated by a peak separation method using a model function, and thus it is difficult to verify whether or not separation degree of each component peak in analysis data obtained by actually performing analysis is desired separation degree.

The present inventors have considered that if what kind of three-dimensional chromatogram data is obtained can be accurately known in advance according to concentration of each of a plurality of components contained in a sample and separation degree of peaks of these components, it becomes possible to verify whether actually obtained analysis data is data having desired separation degree.

Therefore, an object of the present invention is to acquire simulation data of a three-dimensional chromatogram for a sample containing a plurality of components at desired concentration at desired separation degree.

A data processing method according to the present invention is a data processing method for creating simulation data of a three-dimensional chromatogram for a sample containing a plurality of components. The data processing method includes a data preparing step of preparing chromatogram data and spectrum data of each of a plurality of the components, a parameter determining step of determining a parameter including a ratio of concentration of a plurality of the components in the sample to each other and separation degree of peaks of a plurality of the components on a chromatogram from each other, a data adjusting step of adjusting a peak area and a peak position of chromatogram data of each of a plurality of the components based on the parameter determined in the parameter determining step, and a matrix product step of calculating matrix product of a spectrum data group including spectrum data of each of a plurality of the components and a chromatogram data group including chromatogram data of each of a plurality of the components adjusted in the data adjusting step, and creating simulation data of a three-dimensional chromatogram for the sample based on a result of the calculation.

A data processing system according to the present invention is a data processing system that creates simulation data of a three-dimensional chromatogram for a sample containing a plurality of components. The data processing system includes a data storage part that stores chromatogram data and spectrum data of each of a plurality of the components, and an arithmetic processor that performs arithmetic processing using the data stored in the data storage part. The arithmetic processor is configured to execute a parameter determining step of determining a parameter including a ratio of concentration of a plurality of the components in the sample to each other and separation degree of peaks of a plurality of the components on a chromatogram from each other based on information input by a user, a data adjusting step of adjusting a peak area and a peak position of chromatogram data of each of a plurality of the components based on the parameter determined in the parameter determining step, and a matrix product step of calculating matrix product of a spectrum data group including spectrum data of each of a plurality of the components and a chromatogram data group including chromatogram data of each of a plurality of the components adjusted in the data adjusting step, and creating simulation data of a three-dimensional chromatogram for the sample based on a result of the calculation.

In the data processing method according to the present invention, chromatogram data of each of a plurality of components is adjusted by parameters including concentration and separation degree of each of a plurality of the components, and the matrix product of an adjusted chromatogram data group and a spectrum data group is calculated to acquire simulation data of a three-dimensional chromatogram. Therefore, simulation data of a three-dimensional chromatogram when analysis is performed so as to obtain desired separation degree for a sample containing a plurality of components at desired concentration is obtained.

In the data processing method according to the present invention, chromatogram data and spectrum data of a plurality of components are prepared, and, only by determining parameters including concentration and separation degree of each of a plurality of the component, simulation data of a three-dimensional chromatogram corresponding to the determined parameters is acquired. Therefore, it is possible to easily acquire simulation data of a three-dimensional chromatogram when analysis is performed so that desired separation degree is obtained for a sample containing a plurality of components at desired concentration.

### BRIEF DESCRIPTION OF THE DRAWINGS

FIG. 1 is a diagram for explaining a concept of a data processing method;

FIG. 2 is a schematic configuration diagram illustrating an embodiment of the data processing system;

FIG. 3 is a flowchart illustrating a procedure of data processing executed in the embodiment; and

FIG. 4 is a graph showing a verification result of the data processing of the embodiment.

### DETAILED DESCRIPTION OF THE INVENTION

Hereinafter, an embodiment of a data processing method and a data processing system according to the present invention will be described with reference to the accompanying drawings.

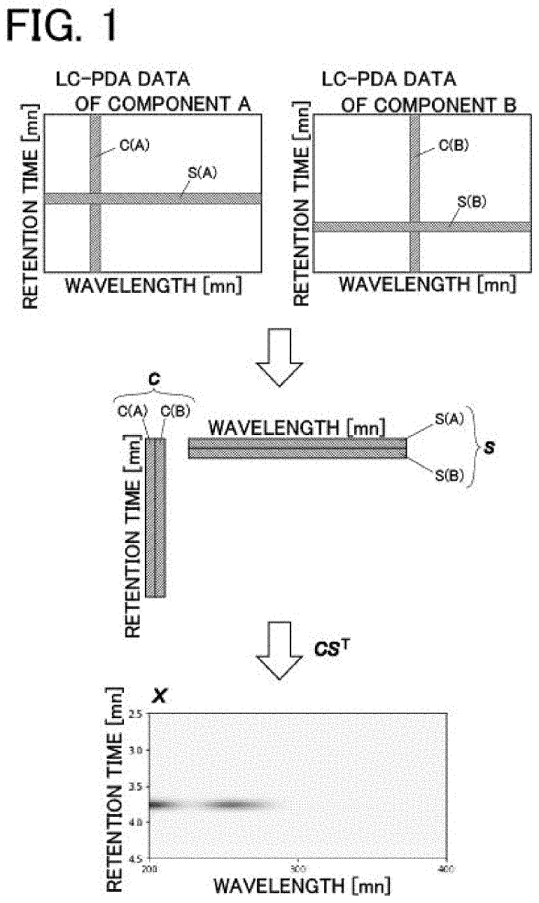

First, a concept of data processing in this embodiment will be described with reference to FIG. 1.

FIG. 1 illustrates a concept for creating simulation data of a three-dimensional chromatogram that is likely to be obtained in a case where a sample containing two components A and B are analyzed by LC-PDA.

In order to acquire target simulation data, it is necessary to prepare chromatogram data C(A) and spectrum data S(A) of the component A, and chromatogram data C(B) and spectrum data S(B) of the component B. The chromatogram data C(A) and C(B) and the spectrum data S(A) and S(B) of each of the components A and B can be created by separately analyzing the components A and B by LC-PDA, or analyzing a sample containing the components A and B under an analysis condition such that peaks of the components A and B are completely separated from each other, and cutting out a wavelength and retention time by which chromatogram information and spectrum information of each of the components A and B can be read from three-dimensional chromatogram data obtained by the analysis.

When a matrix of a chromatogram data group including the prepared chromatogram data C(A) and C(B) of the components A and B is set as
C,

a matrix of a spectrum data group including the spectrum data S(A) and S(B) is set as
S,

and a matrix of three-dimensional chromatogram data obtained by analyzing the components A and B by LC-PDA is set as
X,

the following equation below holds:

CS^{T}=X

where T is the transpose of the matrix. Therefore, simulation data of a three-dimensional chromatogram for a sample containing the components A and B can be obtained by calculating the matrix product

CS^{T}=X

of the matrix of the chromatogram data group
C

and the matrix of the spectrum data group
S.

Here, before the matrix product is calculated, a peak area in chromatogram data of each of the components A and B can be adjusted so that concentration of each of the components A and B contained in the sample becomes a desired value, and a position of a peak in the chromatogram data of each of the components A and B can be adjusted so that separation degree of peaks of the components A and B becomes a desired value. Then, if the calculation of the matrix product is performed using the adjusted data, it is possible to obtain simulation data of a three-dimensional chromatogram when analysis is assumed to be performed under a separation condition that peaks of the components A and B are separated from each other at desired separation degree for a sample containing the components A and B at desired concentration.

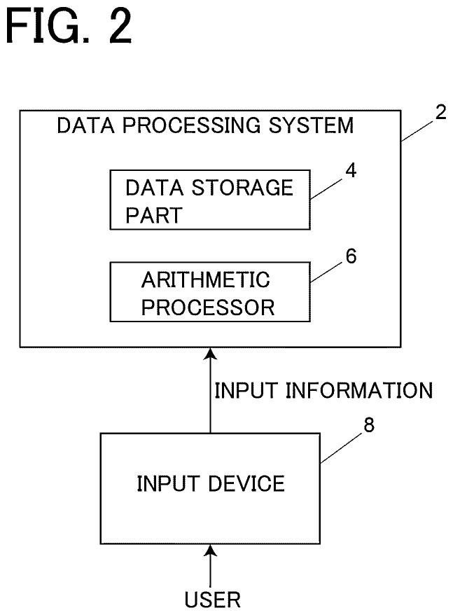

Next, an embodiment of a data processing system for executing the data processing will be described with reference to FIG. 2.

A data processing system 2 includes a data storage part 4 and an arithmetic processor 6. The data processing system 2 can be constructed by introducing a predetermined computer program into a computer device such as a personal computer including a central processor (CPU) and a data storage device such as a hard disk drive or a flash memory. The data storage part 4 is a function implemented by a partial storage area of the data storage device, and the arithmetic processor 6 is a function implemented by the CPU executing a predetermined program.

The data storage part 4 stores chromatogram data and spectrum data of each of a plurality of components included in a sample for which simulation data of a three-dimensional chromatogram is to be created. The chromatogram data and the spectrum data of each of a plurality of components stored in the data storage part 4 may be cut out from three-dimensional chromatogram data acquired by LC-PDA as described above, or may be read out from a database connected to the data processing system 2 via a network. Further, the data processing system 2 may have a function of cutting out chromatogram data and spectrum data of each of a plurality of components from three-dimensional chromatogram data acquired by LC-PDA.

The arithmetic processor 6 is configured to create simulation data of a three-dimensional chromatogram using data stored in the data storage part 4. In creating simulation data, the arithmetic processor 6 determines parameters such as concentration of each of a plurality of components contained in a sample and separation degree between peaks of these components on the basis of information input by the user via an input device 8 such as a keyboard and a mouse, and adjusts a peak area and a peak position of chromatogram data of each of a plurality of components on the basis of the parameters. That is, each value of a matrix group C of chromatogram data is changed or a position of the value is adjusted. Then, the arithmetic processor 6 calculates the matrix product of a matrix including a data group of the adjusted chromatogram and a matrix including a data group of a spectrum to create simulation data of the three-dimensional chromatogram in a state in which peaks of a plurality of components are separated at separation degree designated by the user for a sample in which the components are contained at concentration designated by the user.

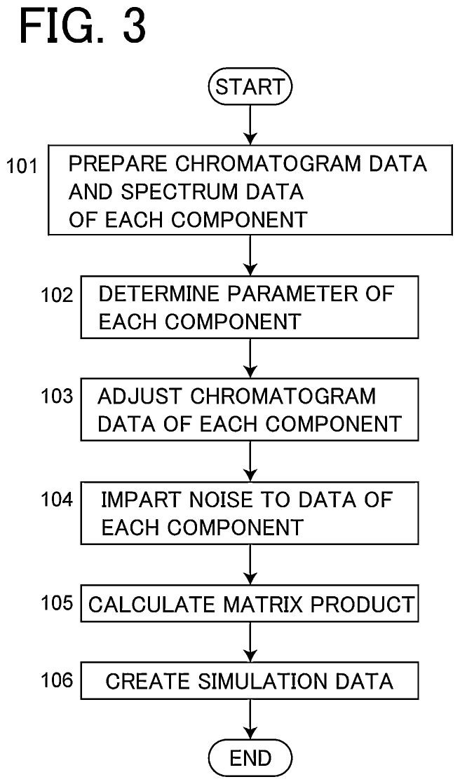

That is, the simulation data of a three-dimensional chromatogram is created by a procedure shown in FIG. 3.

First, chromatogram data and spectrum data of each of a plurality of components included in a sample are prepared (stored in the data storage part 4) (Step 101). Parameters such as concentration of each component in the sample and separation degree of each component are input by the user and determined (Step 102). When parameters are determined, the arithmetic processor 6 adjusts chromatogram data of each component so as to obtain a peak area and a peak position according to the determined parameters (Step 103).

Next, in a case where the user desires, or at all times, the arithmetic processor 6 imparts predetermined noise to each piece of data (Step 104). Noise can be imparted to the adjusted chromatogram data and/or the spectrum data. Examples of the noise to be imparted include one that is randomly generated, as well as a noise component measured when blank liquid is caused to flow through an analysis channel of LC. The user may be able to select a type of the noise to be imparted.

After the above, the arithmetic processor 6 is caused to calculate the matrix product of a matrix of a chromatogram data group including the adjusted chromatogram data and a matrix of a spectrum data group including the spectrum data (Step 105), and further, the arithmetic processor 6 is caused to create simulation data of a three-dimensional chromatogram using a result of the calculation (Step 106).

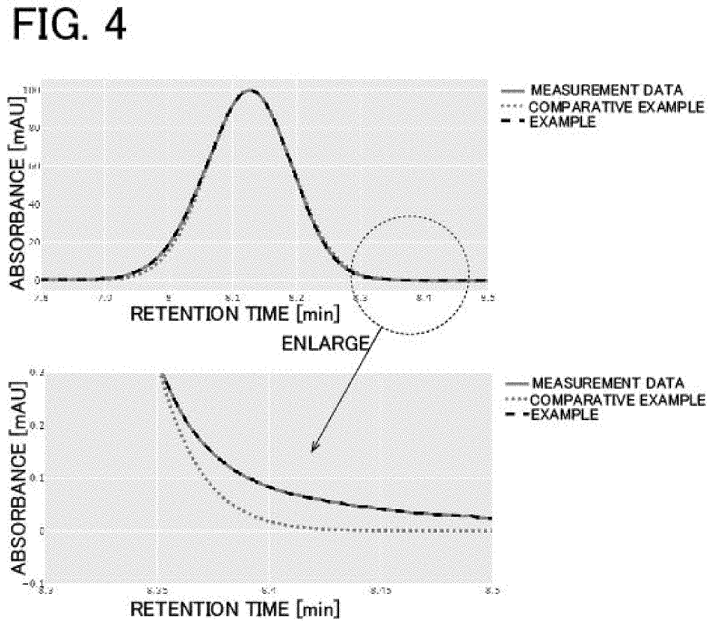

FIG. 4 illustrates a chromatogram at a certain wavelength showing a verification result of simulation data created for a sample containing two components whose concentration ratio with each other is 100:0.05. In this verification, simulation data (example) created using chromatogram data and spectrum data of each of the two components and simulation data (comparative example) of chromatogram created by synthesizing chromatograms of each of the two components simulated by a model function (Gaussian) are compared with chromatogram data (measurement data) obtained by actually analyzing the sample by LC-PDA.

A result of this verification shows that data of the comparative example reproduced using the model function deviates from the measurement data at both end portions of a peak where the two components overlap. On the other hand, data of the example matches with the measurement data over the entire peak. Therefore, it is possible to create highly accurate simulation data of a three-dimensional chromatogram for a sample containing a plurality of components having an extremely large concentration difference by data processing of the example.

Note that the example described above is merely an example of an embodiment of the present invention. The embodiment of the data processing method, the data processing system, and the computer program according to the present invention is as described below.

An embodiment of the data processing method according to the present invention is a data processing method for creating simulation data of a three-dimensional chromatogram for a sample containing a plurality of components. The data processing method includes a data preparing step of preparing chromatogram data and spectrum data of each of a plurality of the components, a parameter determining step of determining a parameter including a ratio of concentration of a plurality of the components in the sample to each other and separation degree of peaks of a plurality of the components on a chromatogram from each other, a data adjusting step of adjusting a peak area and a peak position of chromatogram data of each of a plurality of the components based on the parameter determined in the parameter determining step, and a matrix product step of calculating matrix product of a spectrum data group including spectrum data of each of a plurality of the components and a chromatogram data group including chromatogram data of each of a plurality of the components adjusted in the data adjusting step, and creating simulation data of a three-dimensional chromatogram for the sample based on a result of the calculation.

In the above embodiment, in the data adjusting step, predetermined noise can be imparted to chromatogram data and/or spectrum data of each of a plurality of the components. In this manner, simulation data closer to actual measurement data can be acquired.

An embodiment of the data processing system according to the present invention is a data processing system that creates simulation data of a three-dimensional chromatogram for a sample containing a plurality of components. The data processing system includes a data storage part that stores chromatogram data and spectrum data of each of a plurality of the components, and an arithmetic processor that performs arithmetic processing using the data stored in the data storage part. The arithmetic processor is configured to execute a parameter determining step of determining a parameter including a ratio of concentration of a plurality of the components in the sample to each other and separation degree of peaks of a plurality of the components on a chromatogram from each other based on information input by a user, a data adjusting step of adjusting a peak area and a peak position of chromatogram data of each of a plurality of the components based on the parameter determined in the parameter determining step, and a matrix product step of calculating matrix product of a spectrum data group including spectrum data of each of a plurality of the components and a chromatogram data group including chromatogram data of each of a plurality of the components adjusted in the data adjusting step, and creating simulation data of a three-dimensional chromatogram for the sample based on a result of the calculation.

In the above embodiment, the arithmetic processor may be configured to impart predetermined noise to chromatogram data and/or spectrum data of each of a plurality of the components in the data adjusting step. In this manner, simulation data closer to actual measurement data can be acquired.

A computer program according to the present invention is configured to be introduced into a computer to cause the computer to function as the data processing system described above.

### DESCRIPTION OF REFERENCE SIGNS

2 data processing system
4 data storage part
6 arithmetic processor
8 input device

What is claimed is:

1. A data processing method for creating simulation data of a three-dimensional chromatogram for a sample containing a plurality of components, the data processing method comprising:
a data preparing step of preparing chromatogram data and spectrum data of each of the plurality of components;
a parameter determining step of determining a parameter including a ratio of concentration of the plurality of components in the sample to each other and separation degree of peaks of the plurality of components on a chromatogram from each other;
a data adjusting step of adjusting a peak area and a peak position of chromatogram data of each of the plurality of components based on the parameter determined in the parameter determining step; and
a matrix product step of calculating matrix product of a spectrum data group including spectrum data of each of the plurality of components and a chromatogram data group including chromatogram data of each of the plurality of components adjusted in the data adjusting step, and creating simulation data of a three-dimensional chromatogram for the sample based on a result of the calculation.

2. The data processing method according to claim 1,
wherein in the data adjusting step, predetermined noise is imparted to chromatogram data and/or spectrum data of each of the plurality of components.

3. A data processing system that creates simulation data of a three-dimensional chromatogram for a sample containing a plurality of components, the data processing system comprising:
a data storage part that stores chromatogram data and spectrum data of each of the plurality of components; and
an arithmetic processor that performs arithmetic processing using the data stored in the data storage part, wherein
the arithmetic processor is configured to execute:
a parameter determining step of determining a parameter including a ratio of concentration of the plurality of components in the sample to each other and separation degree of peaks of the plurality of components on a chromatogram from each other based on information input by a user;
a data adjusting step of adjusting a peak area and a peak position of chromatogram data of each of the plurality of components based on the parameter determined in the parameter determining step; and
a matrix product step of calculating matrix product of a spectrum data group including spectrum data of each of the plurality of components and a chromatogram data group including chromatogram data of each of the plurality of components adjusted in the data adjusting step, and creating simulation data of a three-dimensional chromatogram for the sample based on a result of the calculation.

4. The data processing system according to claim 3, wherein the arithmetic processor is configured to impart predetermined noise to chromatogram data and/or spectrum data of each of the plurality of components in the data adjusting step.

5. A computer program configured to be introduced into a computer to cause the computer to function as the data processing system according to claim 3.
